(19) United States
(12) Patent Application Publication
(10) Pub. No.: US 2007/0148677 A1
(43) Pub. Date: Jun. 28, 2007

(54) Methods and systems for acquiring real-time quantitative melt data

(76) Inventors: Alexander M. Chagovetz, Salt Lake City, UT (US); Steven M. Blair, Salt Lake City, UT (US)

Correspondence Address: TRASK BRITT, P.O. BOX 2550, SALT LAKE CITY, UT, 84110, US

(21) Appl. No.: 11/633,980

(22) Filed: Dec. 4, 2006

Related U.S. Application Data
(60) Provisional application No. 60/741,688, filed on Dec. 2, 2005.

(51) Int. Cl. C12Q 1/68; G06F 19/00

(52) U.S. Cl. 435/6; 702/20

(57) ABSTRACT

Described are methods and systems for acquiring real-time quantified melt data. A first Double-stranded nucleic acid are immobilized on a support. The temperature of the double-stranded nucleic acids is slowly ramped up until the double-stranded nucleic acids melt. Differences in fluorescence emission are used to signal when the melt occurred. An evanescent field is used to generate fluorescence emission.

### CROSS-REFERENCE TO RELATED APPLICATION

This application claims the benefit of the filing date of U.S. Provisional Patent Application 60/741,688 filed on Dec. 2, 2005, the contents of the entirety of which is incorporated by this reference.

### TECHNICAL FIELD

The invention relates generally to biotechnology, and more particularly to the field of diagnostics, such as acquiring real-time quantitative melt data for nucleic acids.

### BACKGROUND OF THE INVENTION

Deoxyribonucleic acid (“DNA”) microarrays have emerged as a technology of choice for the purpose of quantifying the expression profiles of ribonucleic acid (“RNA”) libraries. There are numerous methodological approaches to design and manufacture DNA arrays disclosed in the art. However, current methods of quantitative analysis of microarray experiments are generally bound to end-point data acquisitions. This approach is not suited for obtaining reliable quantifications, because of the problems with changing (and often low) signal-to-noise ratios. In order to improve quality of quantitative results, numerous statistical methods have been proposed and applied. Although statistical analysis can resolve some issues with improving signal-to-noise ratios, it cannot address a very critical question, namely what is the contribution of the signal from specific targets in the overall acquired signal.

Melting analysis of polymerase chain reaction (“PCR”) products is a known in the art qualitative method to identify multiple alleles (DNA species) in the sample. It is usually performed as a post-amplification analysis, although there are some considerations for performing this analysis in the middle of the run.

U.S. Pat. No. 6,589,740, filed Mar. 9, 2001, the contents of the entirety of which are incorporated by this reference, discloses detecting hybridization on a biochip while supplying a washing solution over the biochip.

U.S. Pat. No. 6,416,951, filed Mar. 3, 1999, the contents of the entirety of which are incorporated by this reference, discloses detecting hybridization by detecting fluorescence emission excited by an evanescent wave generated near the surface of a wave guide.

U.S. Pat. No. 6,416,951, filed Mar. 3, 1999, the contents of the entirety of which are incorporated by this reference, discloses detecting hybridization by detecting fluorescence emission excited by an evanescent wave generated near the surface of a wave guide.

Stimpson, D. I. et al., “Real-time detection of DNA hybridization and melting on oligonucleotide arrays by using optical wave guides,” PNAS USA, vol. 92, pp. 6379-6383 (1995), the contents of the entirety of which are incorporated by this reference, discloses detecting hybridization by detecting light scattering from an evanescent wave generated near the surface of a wave guide.

### SUMMARY OF THE INVENTION

Certain embodiments of the invention segregate specific target strands from non-specific signals. Additionally, target strand specificity can be validated. The accuracy of relative quantification of the target strands can be enhanced and polymorphisms quantification performed in one step in a massively parallel format. The invention may also provide for chip-based micro-arrays and total analysis without the necessity of PCR.

Certain embodiments of the invention involve a method including fluorescently marking an immobilized double-stranded nucleic acid. An evanescent field is generated in proximity to the immobilized double-stranded nucleic acid and configured to cause fluorescence of the fluorescent marking. The immobilized double-stranded nucleic acid is melted. The intensity of any fluorescence by the fluorescent marking is measured.

Certain embodiments of the invention include a method of acquiring real-time quantitative melting data. The method includes fluorescently marking a double-stranded nucleic acid. The double-stranded nucleic acid is immobilized on a support. An evanescent field is generated in proximity to the immobilized double-stranded nucleic acid and configured to cause fluorescence of the fluorescent marking. The immobilized double-stranded nucleic acid is melted. The intensity of any fluorescence by the fluorescent marking is measured.

Certain embodiments of the invention include a system for quantifying nucleic acid melting. The system include a support and double-stranded nucleic acids immobilized on a surface of the support. Fluorophores are operably coupled to the double-stranded nucleic acids. Excitation equipment is orientated, configured, and located to limit excitation light to within about 100 nm of the surface of the support and to generate light capable of exciting the fluorophores. Detection equipment is orientated, configured, and located to detect fluorescence by the fluorophores.

### BRIEF DESCRIPTION OF THE SEVERAL VIEWS OF THE DPAWINGS

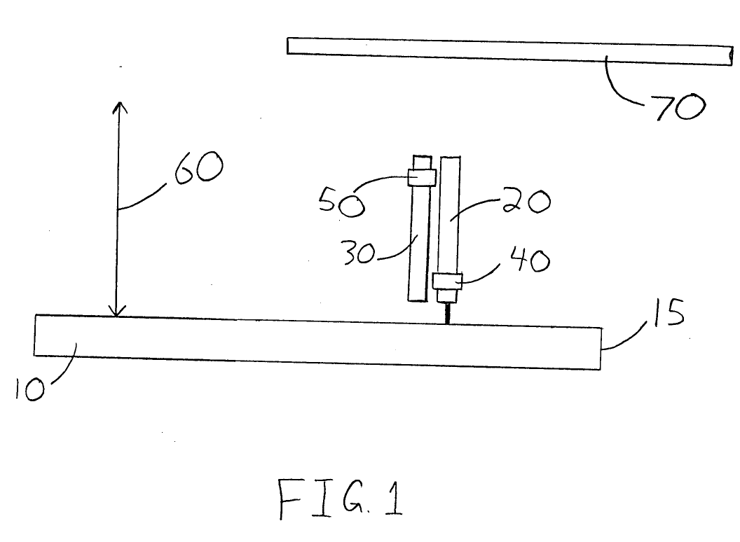

FIG. 1 illustrates a system for quantifying nucleic acid melting.

### DETAILED DESCRIPTION OF THE INVENTION

The invention includes methods and systems for acquiring real-time melting data for double-stranded nucleic acids immobilized on solid supports. Certain embodiments of the invention include a method of quantification of nucleic acids by applying algorithms based on thermodynamic or kinetic models of association and dissociation of double-stranded nucleic acids. Certain embodiments of the invention provide for segregating signals from specific targets from the non-specific signals, validate the target specificity, enhance the accuracy of relative quantification of the targets and perform polymorphisms quantification in one step.

Certain embodiments of the invention include a method of acquiring real-time quantitative melting data. The method includes fluorescently marking a double-stranded nucleic acid. The double-stranded nucleic acid may be immobilized on a support. An evanescent field may be generated in proximity to the immobilized double-stranded nucleic acid and configured to cause fluorescence of the fluorescent marking. The immobilized double-stranded nucleic acid may then be melted. The intensity of any fluorescence by the fluorescent markings may be measured.

Certain embodiments of the invention include a method of identifying specific targets. The method includes slowly ramping up the temperature of a plurality of DNA samples and determining the temperature at which one of the plurality of DNA samples has melted. Then comparing the temperature at which the one of the plurality of DNA samples has melted with known melting points of known DNA samples to determine the identity of the one of the plurality of DNA samples that has melted. The method may be used to identify a polymorphism and specifically SNPs.

Reference will now be made to the figure. Elements in the figure are illustrative only and are not necessarily drawn to scale or indicative of actual geometry.

Double-stranded nucleic acids may be fluorescently marked in a number of ways. The fluorescent marking may be such that intensity of the fluorescence differs after the double-stranded nucleic acid is melted.

FIG. 1 illustrates fluorescent markers 40 and 50. Fluorescent markers 40 and 50 may include fluorophores. Fluorophores can bind to double- or single-stranded species of nucleic acids based on the common structural features of nucleic acids, and which have different fluorescence efficiency (quantum yields of fluorescence) depending on the bound or unbound (free) state of a fluorophore. The fluorescent molecules (fluorophores) can be detected by illumination with light of an appropriate frequency. Light excites the fluorophores and produces a resultant emission spectrum that can be detected by electro-optical sensors or light microscopy.

Intercalating dyes are one type of fluorophore. Intercalating dyes are capable of binding between the stacked planes of nucleobases when two strands of nucleic acids hybridize correctly. The spaces between stacked bases provide a hydrophobic environment. Intercalating dyes are fluorescent markers whose fluorescence is quenched in a polar environment, (i.e., when the dye is in an aqueous solution), but in a hydrophobic environment fluorescence is detectable. Intercalation between nucleic acid strands is accompanied by a change in some property of the dye molecule detectable by optical spectrometry such as a shift in the fluorescence emission frequency of the dye molecule. Therefore, when two nucleic acid strands are hybridized, dye fluorescence is detectably increased. When nucleic acid strands are melted or otherwise dissociated, then the intensity of dye fluorescence is decreased and possibly undetectable. Fluorescence markers 40 and 50 may be intercalating dyes. Therefore, the point at which nucleic acid strands 20 and 30 melt may be detected by exciting the intercalating dye and monitoring the decrease in fluorescence intensity.

Non-limiting examples of intercalating dyes include SYBR GREEN I® (Molecular Probes, Oreg.), ethidium bromide, thiazole orange, oxazole yellow, and respective homodimers. The intercalating dyes may be linked to a nucleic acid strand in a variety of positions with an alkyl linker. Any method of linking intercalating dyes to a nucleic acid strand may be used. Intercalating dyes may be linked to both nucleic acid strands or to just one strand.

Fluorescence resonance energy transfer (“FRET”) labels may also be used to mark the double-stranded nucleic acids. FRET occurs between a donor fluorophore and an acceptor dye, which may be a fluorophore, when the donor fluorophore has an emission spectrum that overlaps the absorption spectrum of the acceptor dye, and the donor fluorophore and acceptor dye are in sufficiently close physical proximity. When light excites the donor fluorophore, there is then produced an emission of light that may be absorbed and quenched by the acceptor molecule. When quenching occurs, the intensity of the donor fluorophore's emission appears to be lessened. Where the acceptor is also a fluorophore, the intensity of its fluorescence may be enhanced. The efficiency of energy transfer is highly dependent on the distance between the donor and acceptor, and equations predicting these relationships have been developed by Forster. FRET is a function of the distance between the donor and acceptor molecules. A discussion of these relationships and Forster—type equations is found in K. Parkhurst and L. Parkhurst, Donor—Acceptor Distance Distributions in a Double-Labeled Fluorescent Oligonucleotide both as a Single Strand and in Duplexes, 34 Biochemistry 1995 pp. 293-300, the contents of the entirety of which are incorporated by this reference.

In a FRET embodiment, fluorescent marker 40 on first nucleic acid strand 20 may be a donor fluorophore. Fluorescent marker 50 on second nucleic acid strand 30 may be labeled with an acceptor dye. The first and second nucleic acid strands 20 and 30 may be labeled such that when the strands are hybridized the fluorescent markers 40 and 50 are sufficiently physically close that detectable FRET can occur. Likewise, when nucleic acid strands 20 and 30 are melted, the distance between fluorescent markers 40 and 50 would increase. Therefore, FRET would either cease or be sufficiently reduced to cause a detectable change in signal intensity. Thus, the point at which nucleic acid strands 20 and 30 melt could be detected by exciting fluorescent marker 40 and monitoring the fluorescence intensity. Alternatively, fluorescent marker 50 may be the donor fluorophore. Additionally, both fluorescent markers 40 and 50 may be a fluorophore.

Non-limiting examples of FRET labels include small organic dye molecules, such as fluorescein, Texas red, or rhodamine, which can be readily conjugated to nucleic acid strands. Additionally, CY™3, CY™5, and CY™5.5 fluorophores may be used. Any type of FRET label may be used.

Fluorescent markers 40 and 50 are illustrated proximate the ends of nucleic acid strands 20 and 30; however, fluorescent markers 40 and 50 may be located anywhere on the strands. Additionally, additional fluorescent markers may be present. Alternatively, either one of fluorescent markers 40 and 50 may not be present. Fluorescent markers 40 and 50 may be linked to nucleic acids 20 and 30 by any method compatible with the nature of the markers.

Any type of double-stranded nucleic acid may be immobilized on the support. Exemplary nucleic acids include genomic deoxyribonucleic acid (“DNA”), messenger RNA, ribosomal RNA, and viral RNA. Additionally, nucleic acid samples of other origin and structure, which may be physically or chemically modified to allow them to be distinguished by fluorescence may also be used. Fluorescent marking may occur before, during, or after immobilization of the double-stranded nucleic acid on the support.

Immobilizing the double-stranded nucleic acid may include immobilizing only a first nucleic acid strand 20 to a support 10 as shown in FIG. 1. The second nucleic acid strand 30 may only be bound by hydrogen bonding to the first nucleic acid strand 20. The first nucleic acid strand 20 may be immobilized by chemical bonds, such as covalent bonds. Alternatively or in addition, affinity and mechanical interactions may also be used. The first nucleic acid strand 20 may be bonded to linking molecules that are in turn immobilized on the support 10. Hybridizing of the first nucleic acid strand 20 and second nucleic acid strand 30 may occur before, during, or after immobilization on the support 10. Any method for immobilizing nucleic acids on a support may be used. It should be understood that a micro-array of double-stranded nucleic acids may be immobilized on the support 10.

An exemplary support 10 is an optical wave guide. Examples of optical wave guides include glass slides and quartz slides. Other substrates known in the art may be used as optical wave guides. Another exemplary support 10 is an etched fiber-optic bundle. Optical wave guides will be used in the discussion below; however, this should not be construed as limiting the invention to optical wave guides. Any support compatible with keeping excitation light from effectively extending beyond approximately 100 nm of the support may be used.

An evanescent field 60 may be generated with the optical wave guide 10 to keep excitation light from extending beyond approximately 100 nm of the optical wave guide 10. The evanescent field 60 is generated by introducing light into edge 15 of the optical wave guide 10 and propagating the light by total internal reflection (i.e., a zig-zag pattern within the optical wave guide 10). Light may be introduced into edge 15 by known methods. For example, a light source such as a lamp in conjunction with a narrow slit in a blind may be used to only introduce light into edge 15. Other exemplary methods include the use of lasers. An evanescent wave (not shown) is created on a surface of the optical wave guide 10. The evanescent wave in turn generates an evanescent field 60. The evanescent field 60 only effectively extends approximately 100 nm from the surface of the optical wave guide 10. Thus, fluorescent markers 40 and 50 (i.e., capable of being excited by the evanescent field) within approximately 100 nm of the optical wave guide 10 will be excited by the evanescent field 60. While fluorescent markers 50 more than 100 nm from the optical wave guide 10 will not be excited by the evanescent field 60. Any method and/or system for generating evanescent fields may be used.

Thus, when nucleic acid strands 20 and 30 are immobilized on the surface of the optical wave guide 10 and an evanescent field 60 generated, fluorescent markers 40 and 50 (assuming both are designed to absorb the wavelength generated) will be excited and detectably fluoresce. Nucleic acid strands 20 and 30 may be in solution (e.g., as a micro-array spot) on the optical wave guide 10. Upon melting, nucleic acid strand 30 will be dissociated. Nucleic acid strand 30 will diffuse into the solution and likely leave evanescent field 60. Therefore, fluorescent marker 50 will no longer be excited and/or participate in a FRET. Fluorescent markers 40 and 50 will have different fluorescent intensities before and after melting. Continuously measuring fluorescence intensity with detector 70 may be used to determine at what point the nucleic acid strands 20 and 30 melted. Detector 70 may be a CCD camera or any other optical detection means compatible with monitoring fluorescence.

The surface of the optical wave guide 10 may also be continuously washed to remove dissociated nucleic acid strands 30.

This analysis is based on the fact that short fragments of double-stranded DNA (“dsDNA”) have “signature melting temperatures.” Depending on the sequence of dsDNA and its length, and also on composition of the environment (ionic strength, stabilizing or destabilizing additives, etc.) the melting point of the DNA species can be predicted, based on thermodynamic calculations. The melting point can be experimentally determined by analyzing a fluorescence signal as a function of temperature (time) during temperature ramps.

The thermodynamic definition of the melting point is unambiguous: melting point is the temperature at which ΔG (measure of thermodynamic stability) is 0, in other words, there is equal thermodynamic stability between single-stranded nucleic acid (“ssDNA”) and dsDNA.

Under quasi-equilibrium conditions, i.e., very shallow temperature ramp, it can be assumed, that at each acquisition point distribution of ssDNA/dsDNA is close to theoretical equilibrium. Hence, the signal change can be predicted based on thermodynamics, if the signal is generated by DNA proper (hypochromism @ 260 nm).

Real-time analysis of the DNA melting on the micro-arrays may be used in identifying polymorphisms using methodology similar to PCR based single nucleotide polymorphism (SNP) detection. Qualitatively, the melting curves obtained in micro-array experiments are similar to homogenous dsDNA melting curves.

dsDNA (PCR product) equilibrium melt using non-specific detectors (e.g., SYBR GREEN I®) or primer based systems. The melt should behave close to thermodynamic predictions, but the information on SNPs or other sequence alterations is very limited due to convergence of the melting points for longer fragments of dsDNA (enthalpy—entropy compensation). In other words, the melt looses its “signature” features, unless the amplicons are very short (preferably less than 60 bp).

In certain embodiments of the invention, where the target strand is labeled with fluorescent dyes (Cy 3, Cy 5, or Cy 5.5, or any other adequate fluorophore), following equilibrium melt does not seem to present a viable option: although fluorescence associated with addressable spots should decline during the melt, presence of dissociated labeled strands in solution would create a high (and changing) fluorescence background, so that the signal to noise ratio would decrease, and so would the sensitivity (accuracy) of quantification. This is not the case, however, if excitation light is held within a very narrow window (˜100 nm) adjacent to the surface of the micro-array (e.g., evanescent field). In case of an evanescent field or under the conditions of total internal reflection (as in etched fiber optics bundles), the presence of detectable moieties in solution does not interfere with signal quantitation on the surface. Consequently, quantitative melting analysis can be performed under quasi-equilibrium approximation, without the necessity of fluidic assembly (flow through to exchange solution). However, multiple melting and annealing events which may be observed in the multi-analyte samples close to equilibrium may result in fluorescence curves (as a function of temperature) which are hard to interpret.

Additionally, an environment for unidirectional (irreversible) melt may be created, where the dissociated strands are removed from the active zone of reaction (micro-array surface) by constant flow of the solution (washing). Under irreversible conditions dissociation rate should follow simple quasi-first order kinetics (quasi relates to the temperature dependence of corresponding rate constants). Integral curve of fluorescence as a function of temperature can be analyzed by applying formalism, based on this basic concept. This approach allows to quantify the amount of target captured by an addressable spot with higher reliability than the end point acquisition: low melting and high melting non-specific signals can be filtered out (i.e., corrected for), while specific melting trace contains multiple acquisitions, and as such is less prone to instrumental artifacts (instability of detector). Finally, the quantity of a bound target can be recalculated in terms of initial concentration in the sample.

Certain embodiments of the invention include a flow through hybridization chamber that has a sidewall of the chamber including a nucleic acid microarray. The chamber includes an active fluidic system, capable of maintaining a variable flow of solution over a surface of the microarray. The chamber also includes a heating system, operable to generate predictable temperature changes on the surface of microarray. The chamber further includes an optical system, capable of generating detectable signals on the surface of microarray. The optical system may utilize fluorescence excitation. The optical system may generate an evanescent field. The optical system may include a detector for quantitative acquisition of generated signals.

Certain embodiments of the invention involve a quantitative method of analyzing fluorescent signals. The method includes analyzing fluorescent signals associated with target nucleic acid strands on the surface of a microarray as a function of temperature (melting analysis), based on the quasi-first order kinetic equation:
dF(fluorescence)/dt=dF/dT*dT/dt=d[dsNA]/dt=k[dsNA]=A·exp(ΔS^{a})·exp(−ΔH^{a}/T)*[dsNA])
wherein A is a collision factor, exp(ΔS) is an temperature independent (entropy of activation) factor, ΔH is an enthalpy of activation factor, and dsNA is a double-stranded nucleic acid.

The method may further include more than one specific target assigned to each fluorescent signal (i.e., polymorphisms) and an algorithm for analyzing multiple species based on the kinetic equation:
dF(T)/dt=ΣᵢdFᵢ(T)/dt.

Certain embodiments of the invention involve analysis based on FRET signal, where the donor fluorescent moieties are placed on or proximal to the 3′ end of immobilized oligonucleotide probes (first nucleic acid strand 20). Acceptor fluorescent moieties are introduced at or proximal to a binding complementary to the probe region (second nucleic acid strand 30), in such a way which favors FRET between donor and acceptor moieties. This method is based on theoretical model of hybridization (2nd order kinetics) reaction, where rate limiting steps are assumed to be diffusion from the volume of the target to the surface address of the probes and chemical (hydrogen bonding) interaction between complementary strands of target nucleic acids (second nucleic acid strand 30) and corresponding oligonucleotide probes (first nucleic acid strand 20). Initial surface concentration of the addressable probes (first nucleic acid strand 20) can be quantified by measuring primary (donor) fluorescence in the absence of target molecules (second nucleic acid strand 30) according to the model:
F(Fluorescence)˜N₁₀˜C₁₀
and reaction rate is described by the mixed diffusion/hybridization chemistry model:
dF₂/dt˜dC₂/dt=k₊₂*C₁*Cₜ−k₋₂*C₂ 1.
dCₜ/dt=h*ΔCₜ+k₋₂*C₂−k₊₂*C₁*Cₜ 2.
dC₁/dt=−k₊₂*C₁*Cₜ+k₋₂*C₂ 3.
where C₁₀ is initial surface concentration (C₁) of the probe, C₂ is surface concentration of the probe/target hybrid, h is an apparent solution diffusion coefficient with assumption of steady-state mixing, unless it is a function of time h(t), Cₜ is a running concentration of the free target molecules in the zone of the reaction, Δ is a Laplasian operator, and F₂ is a secondary (acceptor) fluorescence, associated with formation of a double-stranded hybrid species.

The method may further include more than one acceptor dyes, associated with different target molecules, which can be analyzed by using multi-channel (e.g., spectrometric) detection system.

Certain embodiments of the invention include a method of measuring the relative rate of association or disassociation of two or more types of distinguishable soluble molecules with an immobilized partner molecule. The method may include acquiring data in multiple discrete events over the course of the association or disassociation reaction. Alternatively, the method may includes acquiring data continuously over the course of the association or disassociation reaction, such as from multiple reaction chambers. The soluble molecules may be two or more different samples of genomic DNA, messenger RNA, ribosomal RNA, viral RNA or nucleic acid samples of other origin and structure, which physically or chemically modified to allow them to be distinguished by optical methods. The method may include a device to help normalize data acquisition positional biases when the data acquisition position relative to the association or disassociation reaction changes over time.

While disclosed with particularity, the foregoing techniques and embodiments are more fully explained and the invention described by the following claims. It is clear to one of ordinary skill in the art that numerous and varied alterations can be made to the foregoing techniques and embodiments without departing from the spirit and scope of the invention. Therefore, the invention is only limited by the claims.

What is claimed is:

1. A method comprising:
fluorescently marking an immobilized double-stranded nucleic acid;
generating an evanescent field in proximity to the immobilized double-stranded nucleic acid and configured to cause fluorescence of the fluorescent marking;
causing the immobilized double-stranded nucleic acid to melt; and
measuring intensity of any fluorescence by the fluorescent marking.

2. The method according to claim 1, wherein causing the immobilized double-stranded nucleic acid to melt comprises slowly ramping up the temperature of the immobilized double-stranded nucleic acid.

3. The method according to claim 2, further comprising determining the temperature at which the immobilized double-stranded nucleic acid melted, and comparing that temperature with known melting points of known nucleic acid samples to determine the identity of the immobilized double-stranded nucleic acid.

4. The method according to claim 3, wherein fluorescently marking the immobilized double-stranded nucleic acid comprises fluorescently marking a target strand hybridized to an immobilized probe.

5. The method according to claim 4, further comprising identifying a polymorphism in the target strand.

6. The method according to claim 4, further comprising measuring immobilized probe fluorescence in the absence of target strands according to the model:
F(Fluorescence)˜N₁₀˜C₁₀
and reaction rate is described by the mixed diffusion/hybridization chemistry model:
dF₂/dt˜dC₂/dt=k₊₂*C₁*Cₜ−k₋₂*C₂ 1.
dCₜ/dt=h*ΔCₜ+k₋₂*C₂−k₊₂*C₁*Cₜ 2.
dC₁/dt=−k₊₂*C₁*Cₜ+k₋₂*C₂ 3.
wherein C₁₀ is initial surface concentration (C₁) of the probe, C₂ is surface concentration of the immobilized probe/target strand hybrid, h is an apparent solution diffusion coefficient with assumption of steady-state mixing, unless it is a function of time h(t), Cₜ is a running concentration of the free target molecules in a zone of reaction, Δ is a Laplasian operator, and F₂ is a secondary (acceptor) fluorescence, associated with formation of a double-stranded hybrid species.

7. The method according to claim 4, further comprising identifying a plurality of different target strands using a multi-channel detector.

8. The method according to claim 4, further comprising analyzing fluorescent signals, associated with target strands as a function of temperature based on a quasi-first order kinetic equation comprising
dF(fluorescence)/dt=dF/dT*dT/dt=d[dsNA]/dt=k[dsNA]=A·exp(ΔS^{a})·exp(−ΔH^{a}/T)*[dsNA])
wherein A is a collision factor, exp(ΔS) is an temperature independent (entropy of activation) factor, ΔH is an enthalpy of activation factor, and dsNA is the immobilized double-stranded nucleic acid.

9. The method according to claim 8, further comprising analyzing multiple target strand based on the kinetic equation:
dF(T)/dt=ΣᵢdFᵢ(T)/dT.

10. A method of acquiring real-time quantitative melting data, the method comprising:
fluorescently marking a double-stranded nucleic acid;
immobilizing the double-stranded nucleic acid on a support;
generating an evanescent field in proximity to the immobilized double-stranded nucleic acid and configured to cause fluorescence of the fluorescent marking;
melting the immobilized double-stranded nucleic acid; and
measuring intensity of any fluorescence by the fluorescent marking.

11. The method according to claim 10, wherein fluorescently marking double-stranded nucleic acids comprises incorporating intercalating dyes or fluorescence resonance energy transfer (“FRET”) labels with the double-stranded nucleic acids.

12. The method according to claim 10, wherein immobilizing the double-stranded nucleic acids on a support comprises immobilizing one-strand

13. The method according to claim 10, wherein fluorescently marking the double-stranded nucleic acid comprises fluorescently marking samples of genomic deoxyribonucleic acid (“DNA”), messenger ribonucleic acid (“RNA”), ribosomal RNA, viral RNA or peptide nucleic acid (“PNA”).

14. The method according to claim 10, further comprising acquiring real-time quantitative melting data from multiple supports.

15. A system for quantifying nucleic acid melting, the system comprising:
a support;
double-stranded nucleic acids immobilized on a surface of the support;
fluorophores operably coupled to the double-stranded nucleic acids;
excitation equipment orientated, configured, and located to limit excitation light to within about 100 nm of the surface of the support and to generate light capable of exciting the fluorophores; and
detection equipment orientated, configured, and located to detect fluorescence by the fluorophores.

16. The system of claim 15, further comprising a heater configured and located to control the temperature of the double-stranded nucleic acids immobilized on the surface of the support.

17. The system of claim 15, further comprising a flow system for washing the surface of the support.

18. The system of claim 15, wherein the double-stranded nucleic acids are formed in an array of spots.

19. The system of claim 15, wherein the support comprises an optical wave guide.

20. The system of claim 15, wherein the excitation equipment is configured to generate an evanescent field over the surface of the support.
